(19) United States
(12) Patent Application Publication
(10) Pub. No.: US 2009/0117653 A1
(43) Pub. Date: May 7, 2009

(54) METHOD AND APPARATUS FOR EVALUATING THERAPEUTICS ON CANCER STEM CELLS

(75) Inventors: Julia Kirshner, West Lafayette, IN (US); Linda Pilarski, Spring Lake (CA)

Correspondence Address: SCHWABE, WILLIAMSON & WYATT, P.C.;PACWEST CENTER, SUITE 1900, 1211 SW FIFTH AVENUE, PORTLAND, OR, 97204, US

(73) Assignee: Governors of the University of Alberta, Edmonton, CA (US)

(21) Appl. No.: 12/260,926

(22) Filed: Oct. 29, 2008

Related U.S. Application Data
(60) Provisional application No. 61/000,748, filed on Oct. 29, 2007.

(51) Int. Cl. C12N 5/06; C12M 1/00

(52) U.S. Cl. 435/378; 435/382; 435/307.1

(57) ABSTRACT

Embodiments provide cell culture methods to produce a 3-D model assembly that mimics in vitro the microenvironment of human bone marrow, where cells occupy distinct niches. Methods and apparatuses are provided for the efficient testing of cancer, including malignant hematopoietic cancers and metastatic spread to the bone marrow of solid tumors, and of other diseases of the blood and bone marrow. Methods and apparatuses are further provided in embodiments for the investigation of bone marrow cell biological characteristics and for the testing of therapeutics for nonmalignant diseases of the blood and bone marrow.

### CROSS REFERENCE TO RELATED APPLICATIONS

The present application claims priority to U.S. Provisional Patent Application No. 61/000,748, filed Oct. 29, 2007, entitled “A Novel Method for Evaluation of Therapeutics on Cancer Stem Cells,” the entire disclosure of which is hereby incorporated by reference in its entirety.

All of the references cited within this application are herein incorporated by reference in their entirety to the same extent as if the disclosure of each individual publication, patent application or patent was specifically and individually indicated to be incorporated by reference in its entirety.

### TECHNICAL FIELD

Embodiments herein relate to a method and apparatus for culturing cells, and more specifically, to a method and apparatus for culturing bone marrow cells to mimic the three-dimensional human bone marrow microenvironment for investigation of cancer therapeutics.

### BACKGROUND

Bone marrow (BM) is a soft tissue situated inside hollow bones. As a site of hematopoiesis, BM has a complex organization that includes distinct microenvironmental niches. Discrete extracellular matrix (ECM) microenvironments within the bone marrow help to separate endosteum, an interface between bone and BM, from the central marrow. Various methods exist for studying hematopoiesis, including clonal culture systems in semi-solid media (Pike, B. L. et al., J Cell Physiol, 76, 77 (1970)), and short-term (Golde, D. W. et al., Blood, 41:45 (1973)) and long-term (Dexter, T. M. et al., Biomedicine, 27:344 (1977); Gartner, S. et al., Proc Natl Acad Sci USA, 77:4756 (1980)) liquid culture and tissue culture systems where hematopoietic cells grow on feeder layers of BM stromal cells (Dexter, T. M., et al., J Cell Physiol, 91:335 (1977); Greenberger, J. S., Nature, 275:752 (1978)). However, cell culture systems where cells are grown on the surface of tissue culture plastic fail to accurately represent the three-dimensional (3-D) architecture of the BM, and thus, the complex interactions between cells and their microenvironment.

More recently a stromal spheroid co-culture model (Bug, G., et al., J Leukoc Biol, 72:837 (2002)) and various scaffolds (Niemeyer, P., et al., Cells Tissues Organs, 177:68 (2004); Yang, X. B. et al., Tissue Eng, 10:1148 (2004)) have been developed to re-create the 3-D environment of the BM, but these models fail to recapitulate the physiological conditions of the BM.

Multiple myeloma (MM) is an incurable form of cancer with a projected 3-5 year survival time, despite the development of potent new therapies. MM is characterized by the presence of monoclonal immunoglobulin (Ig) in the blood or urine, lytic bone lesions, and monoclonal plasma cells (PCs) in the BM. Currently, only PCs are evaluated in clinical trials to determine the impact of new drugs, despite the fact that nearly all MM patients relapse, indicating that drug-resistant cancer stem cells escape these therapies. It seems likely that a BM niche maintains MM cancer stem cells (MM-CSC) in a quiescent and drug resistant state.

2-D tissue culture systems of MM cannot sustain proliferation of malignant cells, indicating that the BM microenvironment is essential for the proliferation of the MM clone (Caligaris-Cappio, F., et al., Blood, 77:2688 (1991)). Moreover, there is evidence that the microenvironment influences the effectiveness of therapeutics by mediating drug resistance (Vincent, T. et al., Leuk Lymphoma, 46:803 (2005)). Because clonal expansion of primary MM cells outside their BM microenvironment has been unsuccessful, most pre-clinical studies have resorted to utilizing MM cell lines derived from end stage leukemic phase cells that have escaped BM dependence. Mouse models of MM are equally inadequate for pre-clinical use because they do not faithfully recapitulate the human disease.

### BRIEF DESCRIPTION OF THE DRAWINGS

Embodiments of the present invention will be readily understood by the following detailed description in conjunction with the accompanying drawings. Embodiments of the invention are illustrated by way of example and not by way of limitation in the figures of the accompanying drawings.

FIGS. 1a-d illustrate a 3-D BM culture assembly designed to mimic an in vivo microenvironment of the BM, in accordance with various embodiments; FIG. 1a shows a diagram of a 3-D BM cell culture apparatus designed to mimic bone marrow in vitro; FIG. 1b illustrates cellular composition of a 3-D reconstructed BM (rBM) culture; FIG. 1c illustrates several brightfield microscopic views of a 3-D BM culture demonstrating that BMC grown in rBM recapitulate multicellularity of bone marrow; and FIG. 1d illustrates proliferation of various cellular compartments of rBM after 3-D culture;

FIGS. 2a-d illustrate maintenance of architecture of in vivo BM by rBM in accordance with various embodiments; FIG. 2a illustrates stratification of a 3-D culture; FIG. 2b illustrates redistribution of BM stromal cells to rEnd in a 3-D culture; and FIGS. 2c, d, and e show positions of stem cells (CD34+CD45low), B cells (CD19+/CD33−), and PCs (CD138+/CD38+), respectively, in rBM after 3-D culture;

FIGS. 3a-c show abnormal tissue architecture of MM rBM, which exhibits clonal expansion of MM cells with chromosomal abnormalities observed after 3-D culture in accordance with various embodiments; FIG. 3a shows BMC from normal donors and MM patients grown in rBM in a 3-D culture assembly; FIG. 3b illustrates extent of malignant outgrowth of MM clone in a 3-D culture assembly; and FIG. 3c shows result of fluorescence in situ hybridization (FISH) analysis of cells harvested from a 3-D culture assembly;

FIGS. 4a-d illustrate a comparative evaluation of two MM therapeutics in a 3-D BM cell culture assembly in accordance with various embodiments; FIG. 4a illustrates reduction of clonal cells as measured by RQ-PCR with patient specific primers (melphalan p=0.016, bortezomib p=0.39); FIG. 4b illustrates apoptosis within rBM as assessed by flow cytometry measuring AnnexinV reactivity post treatment with melphalan (left panel) or bortezomib (middle panel). MM specific cell killing by bortezomib was monitored in the CD138+/CD56+ population by AnnexinV reactivity (right panel); FIG. 4c shows cell kill as monitored by brightfield microscopy (200×); and FIG. 4d shows cells at rEnd stained with TRAP (osteoclasts), Oil Red (adipocytes) and ALP (osteoblasts) after removal of the rBM layer;

FIGS. 5a-b show redistribution of cells including putative MM-CSC (label retaining cells, or LRC) to rEnd in a 3-D BM cell culture in accordance with various embodiments; FIG. 5a shows rBM stained with DAPI (blue inset) to detect all cells in the cultures; and FIG. 5b shows cells at rEnd after removal of the rBM layer;

FIGS. 6a-d illustrate stem cell potential of LRCs in a 3-D BM cell culture assembly in accordance with various embodiments; FIG. 6a illustrates a flow cytometry profile of the LRCs; FIG. 6b shows May-Grunwald Giemsa (MGG) staining of sorted LRCs (top) and immunohistochemical staining of sorted LRC for CD20 (bottom); FIG. 6c shows a representative colony from the colony forming unit (CFU) assay; and FIG. 6d illustrates quantification of the LRCs as a percentage of total cells in the culture, CFUs as a percentage of LRCs, and clonal CFUs as a percentage of total colonies obtained in the CFU assay;

FIG. 7 illustrates the importance of serum or plasma from patients with MM or other cancers as a component of 3-D BM reconstruction cultures in accordance with various embodiments; HuN=normal human plasma, HuMM=myeloma plasma or serum, and FBS=fetal bovine serum; and

FIG. 8 illustrates a cell culture assembly in accordance with various embodiments.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, over/under, back/front, and top/bottom. Such descriptions are merely used to facilitate the discussion and are not intended to restrict the application of embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous.

Embodiments provide methods for culturing eukaryotic cells in vitro in an apparatus designed to mimic portions of bone marrow structure such as endosteum and center marrow. In embodiments, a 3-D in vitro culture apparatus allows bone marrow cells to migrate within the apparatus in a manner that more closely resembles their migration in vivo than is possible using traditional culture methods. Embodiments offer an improved opportunity for testing cancer therapeutics on multiple myeloma cells.

Embodiments described herein apply to the investigation of human and veterinary diseases, disorders, cancers, therapies, and/or cell biology of any vertebrate species.

In embodiments, a 3-D in vitro culture apparatus includes a tissue culture vessel coated with a first mixture, a second mixture including a biological matrix mimetic forming a gel over the first mixture, and a cell culture medium overlaying the second mixture. In embodiments, a cancer cell is embedded in the gel or is sandwiched between the first mixture and the gel and the apparatus is incubated. Methods of forming 3-D cell cultures according to embodiments are further described herein.

For the purposes of describing embodiments, the phrase “biological matrix mimetic” refers to any substance/solution/mixture, including a commercially available product such as Matrigel™, that is designed/produced/used to mimic or approximate in vitro one or more biological matrices such as, for example, an extracellular matrix, a basement membrane, and/or a structure of a connective tissue. Similarly, the phrase “cell culture substrate” refers to any substance/solution/mixture, including a commercially available product such as Methocult™, that is designed/produced/used to culture colony-forming cells.

For the purposes of describing embodiments, the term “LRC” or “label-retaining cell” refers to a cell in a state of proliferative quiescence, such as determined by carboxyfluorescein succinimidyl ester (CFSE) labeling; labeled cells exhibit fluorescence that decreases in intensity by half each time the cell divides, providing a measure of proliferation. In addition, “putative MM-CSC” refers to quiescent, drug resistant LRCs that are able to generate colonies of clonotypic cells upon entering an active or non-quiescent state.

For the purposes of describing embodiments, the terms “therapy”, “therapeutic”, and their plurals refer to chemicals, compounds, mixtures, solvents, drugs, and any other treatment applied to one or more cells for, or to assess potential for, treatment of cancer and/or to investigate one or more biological characteristics of eukaryotic cells, including both normal cells and abnormal/diseased/cancer cells.

For the purposes of describing embodiments, “cell” or “cells” may refer to any eukaryotic cell, whether human or animal.

For the purposes of describing embodiments, the phrases “tissue culture vessel” and “cell culture vessel” are interchangeable and refer to any vessel/container suitable for the growth of eukaryotic cells, including any vessels/containers commercially available for that purpose. Tissue culture vessels may be constructed from materials including, but not limited to, polystyrene, a polymer, glass, plastic, etc. and may be treated/coated/constructed with a surface adapted for cell attachment. Such surfaces may be hydrophilic, hydrophobic, negatively charged, positively charged, non-ionic, or altered in texture to increase one or more surface areas. In addition, tissue culture vessels may be gas permeable and/or may include a cap/lid/closure that is gas permeable. Tissue culture vessels in accordance with embodiments include, but are not limited to, flasks, single well plates, multi-well plates, microtiter plates, bottles, Petri dishes and other containers.

For the purposes of describing embodiments, the phrase “growth medium” refers to a cell culture medium comprising RPMI with L-glutamine, fluid from a vertebrate with cancer or nonmalignant disease of the bone marrow or blood, 6.2×10⁻⁴M CaCl₂, 1×10⁻⁶M sodium succinate, and 1×10⁻⁶M hydrocortisone. Growth medium may also include an antimicrobial/antibiotic/antifungal substance. The molarity and/or molality of each component in growth medium may vary among embodiments. In addition, “fluid from a vertebrate with cancer or nonmalignant disease of the bone marrow or blood” may include plasma, serum, peritoneal fluid, ascites fluid, cerebrospinal fluid, blood, lymph and/or synovial fluid from any vertebrate animal (including but not limited to humans, non-human primates, rats, mice, rabbits, pigs, dogs, and others) with any form of cancer, including but not limited to multiple myeloma and cancers of bone, soft tissue, muscle, skin and/or blood. For example, in some embodiments illustrated herein, growth medium comprises RPMI with L-glutamine, 20% MM patient plasma, 6.2×10⁻⁴M CaCl₂, 1×10⁻⁶M sodium succinate, and 1×10⁻⁶M hydrocortisone.

Embodiments provide a unique pre-clinical model that supports in vitro expansion of an MM clone and allows access to MM cancer stem cells. As cancer cells are highly drug resistant in 3-D microenvironments, adhesion mediated drug resistance is a confounding factor for conventional MM pre-clinical models. While currently available drugs reduce the tumor burden, thus improving patient survival and quality of life, they do not cure the disease. 3-D reconstructions of BM deliver a powerful model where the effects of therapies may be tested not only with respect to the reduction of the PC tumor mass, but on the entire MM clone, the extent of which is not otherwise well defined. In this model, putative MM-CSC reside at the reconstructed endosteum, the niche known to maintain dormancy of normal HPC. While not all LRCs from 3-D cultures have stem cell potential, harvesting of LRC enriches for and provides access to dormant MM-CSC and, when released from dormancy, their progeny.

While exemplary embodiments illustrated herein describe the use of 3-D apparatuses in the context of MM, embodiments are suitable for, and intended for use in, the study of any bone marrow cell and/or a bone marrow microenvironment. Embodiments may be used to culture cells for the analysis of any hematopoietic cancer of blood and/or bone marrow, nonmalignant diseases of blood and/or bone marrow, metastatic spread of cancers to the bone marrow, and effects of therapies on any cell located in, residing in, and/or originating from bone marrow.

The identity of the MM-CSC remains controversial with some studies suggesting that the disease arises from a PC and others indicating a B-lymphocyte origin of MM. Data obtained from the SCID-hu mouse model of MM suggests that MM arises from a PC (CD38+CD45−) (Yaccoby, S. et al., Blood, 94:3576 (1999)). However, in this model, MM cells are forced to colonize a microenvironment provided by fetal bone which may not support growth of populations that flourish in BM of older adults, as occurs in MM. This concern is supported by the present observation that plasma from young normal donors cannot sustain proliferation of the MM clone in 3-D culture. The evidence for the non-PC (CD138−) origin of MM comes from work in MM cell lines and ex vivo BM samples grown in a conventional 2-D culture and evaluated in a CFU-type of assay. Xenografting of CD34-enriched cells from MM mobilized blood autografts gives rise to lytic bone lesions and clonotypic progeny in murine BM, and early stage clonotypic MM B cells are self-renewing in immunodeficient mice. However, in a murine model it is difficult to resolve the fine details of cell-cell and cell-microenvironment interactions underlying MM. With the development of the 3-D ex vivo culture model that makes proliferating and non-proliferating compartments of MM BM accessible for further analysis and identification of their progeny, the identity, generative capabilities and self-renewal potential of the MM-CSC may be established.

Embodiments disclose culture methods and apparatuses useful for the presently underexplored aspect of pre-clinical testing in which malignant cell expansion is observed within the context of the aggregate microenvironment. The 3-D reconstruction of the BM microenvironment provides an essential tool for evaluating the therapeutic potential of treatment strategies and new drugs on the entire malignant hierarchy. The rBM model may be used to study any cancer and/or disorder of the blood and/or bone marrow, including but not limited to MM, Waldenstrom's macroglobulinemia, acute promyelocytic leukemia, monoclonal gammopathies, leukemias, myeloproliferative disorders, myelodysplastic syndromes, anemias, plasma cell disorders, lymphomas, hemoglobinopathies, and cancers spreading to the bone marrow. Not only may the rBM model be used for pre-clinical testing of therapies for MM and other bone marrow/blood disorders, it provides a comprehensive model to study the basic biology of BM and its diseases. The 3-D ex vivo model is a useful tool to study cytokine/chemokine and growth factor networks in the normal and malignant BM as well as the cellular signaling in normal tissue homeostasis and disease state. The methods taught here may be readily applied to all cancers of the bone marrow because all reside in the bone marrow microenvironment that is reconstructed in 3-D cultures. Moreover, this model may be adapted for high throughput analysis of drugs with potential to target various cellular compartments, including but not limited to characterization of chemotherapeutic effect on solid-phase tumors in the bone, MM, Waldenstrom's macroglobulinemia, acute promyelocytic leukemia, monoclonal gammopathies, leukemias, myeloproliferative disorders, myelodysplastic syndromes, anemias, plasma cell disorders, lymphomas, hemoglobinopathies, and all cancers of the blood and/or bone marrow.

Embodiments provide apparatuses and methods for the preparation and use of eukaryotic cell cultures. An embodiment provides a method for reconstructing a human bone marrow microenvironment in vitro that allows a MM clone to expand. Methods for forming 3-D tissue cultures in accordance with embodiments are also provided for the observation of malignant cell expansion within an assembly that approximates a microenvironment of human BM. Further embodiments provide methods for culturing BM cells and/or mobilized blood autografts for screening, analysis and characterization of therapeutics for multiple myeloma as well as for other cancers of the hematopoietic system and metastatic disease of solid tumors that home to the bone marrow.

In embodiments, a method for forming a 3-D culture of eukaryotic cells includes coating a tissue culture vessel with a mixture including cell adhesion molecules to mimic endosteum, overlaying a mixture that forms a gel to mimic central marrow, overlaying the gel with cell culture media, and incubating the tissue culture vessel. In some embodiments, one or more eukaryotic cells may be disposed between the coating and the gel, while in other embodiments a eukaryotic cell may be embedded in the gel.

More specifically, a method for forming a 3-D culture of eukaryotic cells in accordance with an embodiment comprises coating a portion of an interior surface of a tissue culture vessel with a first mixture, the first mixture comprising fibronectin and collagen; adding a second mixture onto the portion of the interior surface of the tissue culture vessel, the second mixture comprising a eukaryotic cell, fibronectin, and a biological matrix mimetic, wherein the second mixture forms a gel, and wherein the eukaryotic cell is embedded in the gel; adding a third mixture onto a surface of the second mixture, the third mixture comprising a fluid from a vertebrate subject having cancer or other disease of the hematopoietic system or blood/bone marrow systems, including metastatic disease of solid cancers that home to the bone marrow; and incubating the tissue culture vessel, such as at a temperature of approximately 30-45° C., for example at approximately 37° C., and at 1-10% CO₂, for example at approximately 5% CO₂.

In an embodiment, the second mixture further comprises one or more cancer cell-derived substances. In some embodiments, the second mixture includes a fluid from a vertebrate with cancer or nonmalignant disease of the bone marrow or blood. Some embodiments further comprise adding a fluid derived from a vertebrate with cancer or nonmalignant disease of the bone marrow or blood onto the surface of the second mixture, while other embodiments further comprise adding a cell culture fluid and/or diluent onto the surface of the second mixture. One or more cell adhesion factors may also be added to the second mixture and/or to a fluid derived from a vertebrate with cancer or nonmalignant disease of the bone marrow or blood.

In an alternate embodiment, a method for 3-D culture of eukaryotic cells comprises coating a portion of an interior surface of a tissue culture vessel with a first mixture, the first mixture comprising fibronectin and collagen; adding a eukaryotic cell to the portion of the interior surface of the tissue culture vessel; adding a second mixture onto the portion of the interior surface of the tissue culture vessel, the second mixture comprising fibronectin and a biological matrix mimetic, wherein the second mixture forms a gel and wherein the eukaryotic cell is disposed between the first mixture and the gel; adding a third mixture onto a surface of the second mixture, the third mixture comprising a fluid from a vertebrate subject having cancer or nonmalignant disease of the bone marrow or blood systems; and incubating the tissue culture vessel at approximately 37° C. and approximately 5% CO₂. Again, the second mixture may further comprise one or more cancer cell-derived substances and/or fluid from a vertebrate with cancer or nonmalignant disease of the bone marrow or blood, and either or both of these may be added to the surface of the second mixture. One or more cell adhesion factors (see below) may also be added to the first mixture, the second mixture and/or the fluid derived from a vertebrate with cancer or nonmalignant disease of the bone marrow or blood.

While Matrigel™ is the biological matrix mimetic in some embodiments, in other embodiments another biological matrix mimetic and/or one or more components of Matrigel™ may be substituted as the biological matrix mimetic.

Cell adhesion factors are well known in the art and include fibronectin, collagen 1, collagen IV, laminin, vitronectin. tenascin, positively-charged molecules ((such as poly-l-lysine, chitosan, poly(ethyleneimine), polymerized acrylics, etc.)), cell surface carbohydrate-binding proteins/glycoproteins, integrins, fragments/subunits of cell adhesion molecules, synthetic analogs of cell adhesion molecules, gelatin, poly-1-ornithine, etc. Embodiments may include 1, 2, 3, 4, 5, 6, or more of these factors as components of the first mixture and/or the second mixture, and/or include one or more as additives to a fluid derived from a vertebrate with cancer or nonmalignant disease of the bone marrow or blood. In addition, the ratios and/or concentrations of these factors may vary among embodiments.

Additionally, one or more diluents and/or cell culture media may be added to any of the mixtures and/or to the fluid from a vertebrate with cancer or nonmalignant disease of the bone marrow or blood. Diluents/media may include water, phosphate-buffered saline (PBS), RPMI, Growth Medium, Minimal Essential Medium (MEM), Eagle's Basal Medium (BME), Dulbecco's Modified Eagle's Medium (DMEM), Hank's Balanced Salt Solution (HBSS), etc.

In embodiments, cells cultured in a 3-D BM culture assembly may include a bone marrow cell, a putative MM-CSC, and/or a LRC. While certain portions of the present description are directed to culturing MM/cancer cells, embodiments may provide assemblies, apparatuses and methods for culturing any cell type, including but not limited to blasts, polymorphonuclear cells, neutrophils, eosinophils, basophils, pre-PMN cells, promyelocytes, myelocytes, metamyelocytes, lymphocytes, B and/or T cells, nucleated red cells, proerythroblasts, basophilic erythroblasts, polychromatophilic erythroblasts, orthochromatic erythroblasts, macrophages, stromal cells, reticular cells, osteoclastic cells, osteoblastic cells, etc. Similarly, while certain embodiments described with relation to methods/systems/apparatuses for investigating the biological characteristics of, or effects of therapeutics on, MM/cancer cells, other embodiments may also provide corresponding systems/apparatuses/methods for any cell type or any type of cancer found in the bone marrow or blood system, including but not limited to those listed above. Additionally, some embodiments may be adapted for high-throughput screening/analysis by assembling 3-D cultures in microtiter plates with 384 wells, 1536 wells, 3456 wells or some other number of wells.

An embodiment further provides an apparatus that supports in vitro expansion of a MM clone, providing access to MM cancer stem cells for further research. Apparatuses in accordance with embodiments provide a preclinical model for testing the impact of drugs and/or other therapies on a cellular compartment of rBM, a MM clone, a cancer cell, and/or a tumor. An embodiment provides an apparatus for the study of cytokine/chemokine and growth factor networks in normal and malignant BM as well as cellular signaling in normal tissue homeostasis and in disease state tissue homeostasis. Additional embodiments provide apparatuses for high-throughput and/or high-content analysis of therapies with potential to target various cellular compartments of BM, including but not limited to characterization of chemotherapeutic effect on individual cells, normal cells, cancer cells, and solid-phase tumors in the bone. In an exemplary embodiment, an apparatus is provided that allows MM cells to colonize a microenvironment provided by fetal bone, although fetal bone does not typically support the colonization of MM cells in vivo.

Embodiments provide apparatuses for testing a cancer therapy comprising a 3-D tissue culture assembly, an incubator, one or more therapies, and a eukaryotic cell. In an embodiment, the 3-D tissue culture assembly includes a tissue culture vessel; a bottom layer coupled to an interior surface of the tissue culture vessel and comprising fibronectin and collagen; a middle layer disposed over a surface of the bottom layer and comprising a biological matrix mimetic and fibronectin, wherein the middle layer forms a gel; a top layer disposed over a surface of the middle layer and comprising a fluid derived from a vertebrate with cancer or nonmalignant disease of the bone marrow or blood. In embodiments, an incubator is configured to maintain a condition of approximately 37° C. and approximately 5% CO₂, although other temperatures and CO₂ concentrations may be utilized. In an embodiment, a therapy may belong to a group consisting of a chemical, a compound, a drug, a mixture, and a solvent.

In some embodiments, one or more eukaryotic cells may be embedded within the middle gel layer of the 3-D tissue culture assembly, while in other embodiments a eukaryotic cell is disposed between the bottom layer and the middle layer. In an embodiment, a cell of a first type may be embedded within the middle gel layer while a cell of another type may be disposed between the bottom layer and the middle layer. 3-D tissue culture assemblies may be further modified as described for 3-D culture methods above.

In an exemplary embodiment, an apparatus for testing cancer therapies reconstructs bone marrow microenvironments and allows a therapy to be applied to normal and/or cancer cells, which may then be examined to determine the effect of the therapy on the cell. In some embodiments, an apparatus is adapted for high-throughput screening/analysis by decreasing the size/volume of 3-D tissue culture assemblies and/or using microtiter plates with 384 wells, 1536 wells, 3456 wells or some other number of wells. Cultures may be examined by an individual, by a computer, an automated machine, robotically, or by another method. 3-D tissue culture assembly apparatuses may also be adapted for high-content screening and may include an automated imager, a digital microscope/imager, and/or a flow cytometer. Digital microscopes in accordance with various embodiments may be fluorescence microscopes, automated microscopes, confocal microscopes, widefield microscopes, etc. Additionally, embodiments of apparatuses for testing cancer therapies may include software for image analysis.

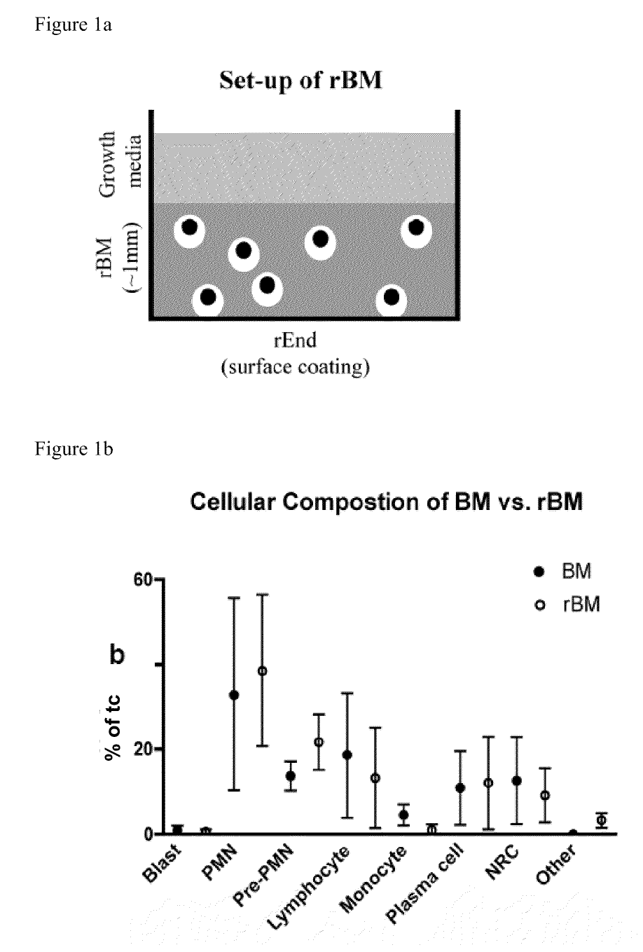

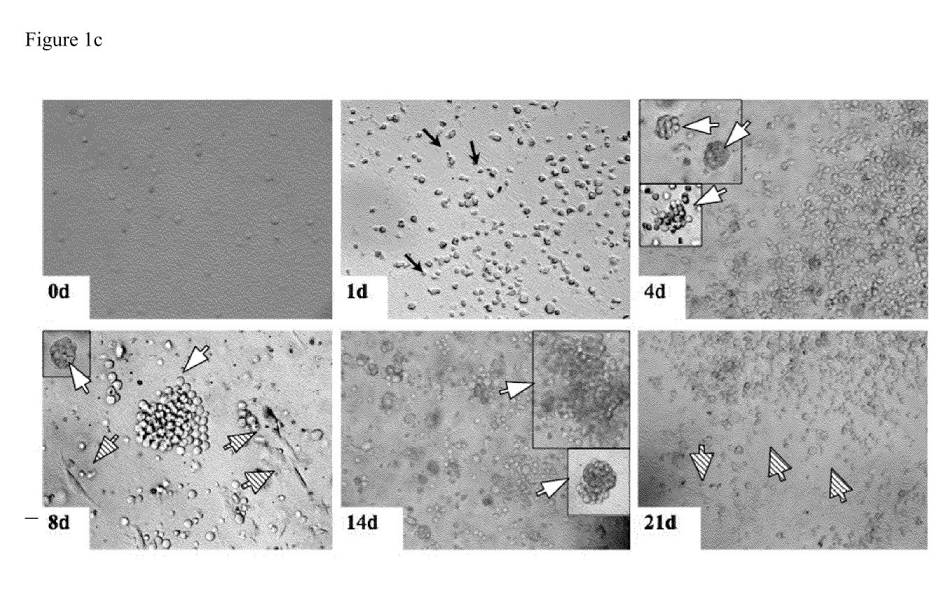

In the embodiments illustrated in FIGS. 1-7, 3-D cell culture and other techniques were performed. BMC were isolated from BM aspirates by Ficoll-Paque gradient centrifugation per manufacturer's instructions. Surface coating (rEnd) was created by incubating tissue culture plates with fibronectin/collagen I (1:1) in PBS at a final concentration of 5 μg/1 cm² of each protein. Plates were incubated for <30 min at room temperature (RT) and the remaining fluid was removed and was replaced with the rBM layer consisting of the BMC cells in an ECM mixture of Matrigel™/fibronectin (2:1 v/v). For analysis of proliferation and LRCs, cells were labeled with 0.25 μM CFSE for 15 min at 40° C. The labeling reaction was stopped with cold PBS for 10 min on ice, cells were centrifuged to remove excess dye, resuspended at 0.5×10⁶ cells/1 cm² in 10 μl of PBS, mixed with 100 μl/1 cm² of ECM mixture and plated on top of the fibronectin:collagen I endosteal coating. The culture was allowed to solidify for 30 min in a 37° C., 5% CO₂ incubator and was overlaid with 1 ml of the Growth Medium (RPMI with L-glutamine, 20% MM or other patient plasma, 6.2×10⁻⁴M CaCl₂, 1×10⁻⁶M sodium succinate, 1×10⁻⁶M hydrocortisone). Serum was found to be an essential growth factor which could not be replaced by serum from healthy donors (in preparation). 3-D cell culture materials included Matrigel™ (BD Biosciences) (˜10 mg/ml protein), composed of 56% laminin (5.6 mg/ml) and 31% collagen IV (3.1 mg/ml); fibronectin (Upstate, 100 MG); Growth medium−RPMI (with additives)+20% plasma; Collagen I (Upstate). Additives used were 6.2×10⁻⁴M CaCl₂; 1×10⁻⁶M Sodium succinate; 1×10⁻⁶M Hydrocortisone; and optionally penicillin/streptomycin.

For co-culture experiments, CD34+CD45low stem cells from mobilized blood autografts, CD19+CD33− B cells and CD38+CD138+ PCs from MM BM were sorted on an EPICS ALTRA cell sorter, Beckman Coulter (Mississauga, ON), labeled with CSFE, mixed with unlabeled BMC and cultured as described above. Cells were isolated from 3-D cultures with Cell Recovery Solution per manufacturer's instructions and analyzed as indicated.

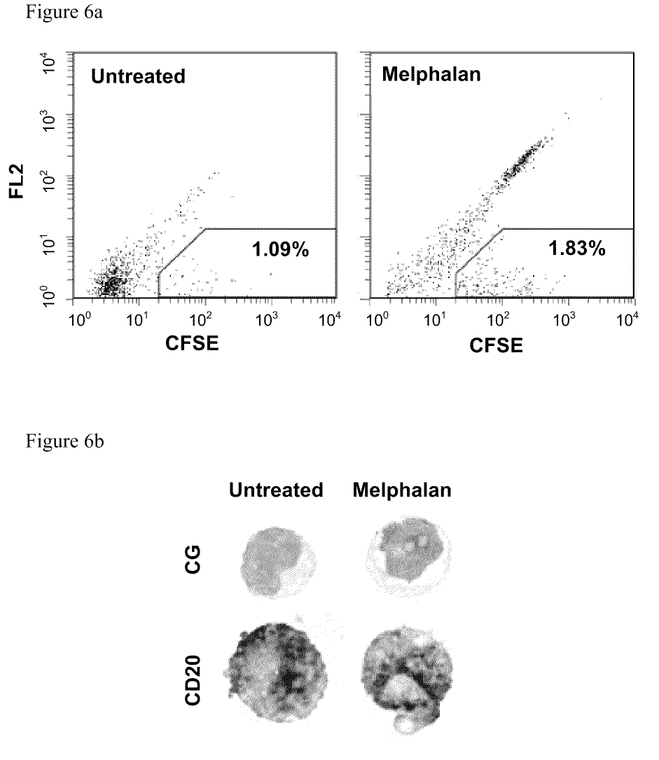

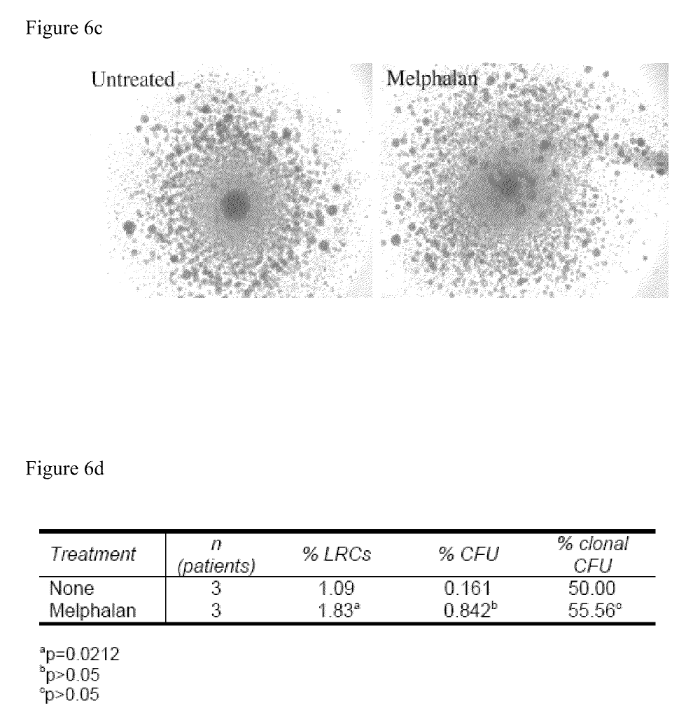

For CFU assays, CFSE labeled cells were isolated from the 3-D culture and the CFSE+ cells were sorted on an EPICS ALTRA cell sorter, by gating on the live cells based on the forward and side scatter and on the CFSE+ population based on fluorescence intensity in the FL1 channel (see FIG. 6a, below). Sorted cells were resuspended in 200 μl IMDM with 2% FBS, mixed with 1.1 ml of MethoCult™ GF medium, and plated in 35 mm plates. The MethoCult™ cultures were incubated for 14 days and the resulting colonies were counted. To isolate the DNA, individual colonies were transferred into PBS and centrifuged to remove MethoCult™ and the pellets were resuspended in TRIzol™, followed by purification of genomic DNA. Cancer stem cells may also be obtained without the need for CFSE and cell sorting by directly transferring all of the cells harvested from a 3-D culture into a set of CFU cultures.

For preclinical studies, the rBM was grown for 14 days to allow for stratification of the culture and expansion of the malignant cells and the stromal layer. A single treatment of either melphalan or bortezomib was administered in a fresh aliquot of Growth Medium overlaid on top of the 3-D culture. After 7 days of treatment, fresh medium was added and cells were allowed to recover for an additional period of 7 days, after which cells were isolated using the Cell Recovery Solution and the effects of treatment were analyzed, and for CFU activity of drug resistant LRC.

For real-time quantitative PCR (RQ-PCR), total genomic DNA was isolated with TRIzol™ reagent (Invitrogen, Burlington, ON) per manufacturer's instructions. RQ-PCR for patient-specific rearranged IgH VDJ clonotypic sequences was performed using the SYBR green method (Li, A. H., et al., Exp Hematol, 30:1170 (2002)) with product specificity confirmed by melting curve analysis, established as a well controlled technique per manufacturer's instructions. Patient specific primers for CDR2 and CDR3 were designed. Because only one allele of the IgH VDJ has the clonotypic gene rearrangement, each malignant cell has only one genomic copy of this molecular MM signature. Controls included cloned VDJ sequences for each patient used to generate a standard curve for the specific clonotypic sequence and comparison with a standard curve for a housekeeping gene, β2 microglobulin (normalized to the two copies of B2M templates/cell), to quantify the number of templates in unknown samples before and after 3-D culture.

For microscopy, confocal z-slices were imaged on a Carl Zeiss confocal LSM 510 microscope equipped with appropriate filters and the 3D reconstruction was done using Zeiss 510 image analysis software. Quantification of LRC positioning was performed with Imaris 3.2.2 software, Bitplane AG (Zurich, Switzerland). Stromal cells were stained with TRAP, Oil Red, Alkaline phosphatase, or Alizarin Red according to the manufacturer's instructions and imaged on Zeiss Axioskop digital microscope. For fibronectin, phalloidin and N-cadherin staining, cells were fixed with 1% neutral buffered formalin for 10 min at RT, permeabilized with 0.5% Triton X-100 and non-specific binding was blocked with 1% BSA for 1 hr at RT. Cells were incubated with primary antibodies, followed by a wash and incubation with secondary antibodies for 1 hr and confocal imaging.

For flow cytometry, cells were isolated from 3-D cultures with Cell Recovery Solution, stained with various FITC tagged CD antigens or Annexin V-PE and analyzed by flow cytometry to measure the extent of proliferation and apoptosis using a FACSort flow cytometer (BD, Oakville, ON). Data was analyzed with CellQuest Pro software.

Fluorescent in situ hybridization (FISH) was performed as follows. Cells pre- and post-culture were immobilized onto a glass microscope slide by cytospinning. The slides were then stained with May-Grunwald for 3 min at RT, washed in H₂O, stained with Giemsa in dH₂O(1:20) for 10 min at RT and rinsed with H₂O. The position of all cells on the slide was recorded using the BioView Duet scanning system (Rehovot, Israel). The slide was then destained in methanol/acetic acid (3:1) for 60 mins, and conventional interphase FISH analysis was performed as described by Sieben et al. (Sieben, V. J., et al., IET Nanobiotechnol, 1:27 (2007)). Analysis of conventional interphase FISH analysis was performed as described (Sieben, V. J., et al., IET Nanobiotechnol, 1:27 (2007)). Three types of chromosomal abnormalities were analyzed: 1) a commercial Vysis LSI®13q34 probe for the detection of the deletion of 13q34 locus, 2) Vysis LSI®IGH/CCND1-XTdual color-dual fusion translocation probe for the detection of translocation (11;14)(q13;q32) and 3) Vysis CEP1(D1Z5) as a control probe combined with a home-made probe targeting locus 1q21 for the detection of the amplification of 1q21. Brightfield and fluorescence microscopy was performed using the Bioview Duet system to correlate the FISH staining pattern and the morphology for each cell on the slide.

For statistical analysis, data were presented as mean±standard error of the mean (SEM). Statistical significance was measured by Student's t-test using Prism 4 software from GraphPad Software, Inc. Differences in the mean values were reported as p-values with p<0.05 considered significant.

Embodiments provide 3-D cell culture assembly including a cell culture vessel, a surface coating to mimic an endosteal matrix, a gel layered over the surface coating to mimic a bone marrow matrix, and growth media overlaid on the surface of the gel.

FIGS. 1a-d illustrate a 3-D BM culture apparatus designed to mimic the in vivo microenvironment of the BM, in accordance with various embodiments.

FIG. 1a illustrates an apparatus for reconstructing an in vivo bone marrow microenvironment in vitro. The illustrated embodiment includes a fibronectin/collagen I surface coating as rEnd, overlaid with a mixture of bone marrow cells (BMC) and Matrigel™/fibronectin ECM, and covered with Growth Medium. In embodiments, the in vitro reconstructed endosteal (rEnd) and BM (rBM) matrices mimic the in vivo composition of endosteum and BM, and may vary in the composition of the surface coating, the gel layer and/or the media overlaid on the surface of the gel. In the illustrated embodiment, rEnd is constructed by coating the surface of the tissue culture plate with a collagen I:fibronectin protein mixture, the rEnd is then overlaid with BM mononuclear cells (BMC) suspended in a mixture of fibronectin and Matrigel™, which is comprised mainly of laminin and collagen IV (this mixture is representative of the in vivo central marrow, composed of fibronectin, laminin, and collagen IV, and forms a gel upon incubation at 37° C.), and the gel is then overlaid with growth media. In embodiments, one or more substitutes for fibronectin, Matrigel™, growth media and other components may be used.

FIG. 1b shows the cellular composition of a 3-D culture constructed as described above. To confirm that the cellular composition of the pre-culture BM and the rBM post-culture were similar, the proportion of each cellular compartment was determined in BMCs and in cells from dissociated 3-D cultures. Cellular composition of a 3-D culture was assessed after 3 weeks using cytospins prepared from the pre-culture BMC (n=6) and from the same samples post-culture (n=6), stained with May-Grunwald Giemsa. Nuclear and cytoplasmic staining and morphology were analyzed and cells of various lineages were counted and reported as percent of total cellularity. Cells counted in FIG. 1b include: blasts, PMN (polymorphonuclear), bands, neutrophils, eosinophils, basophils; pre-PMN, promyelocyte, myelocyte, metamyelocyte; lymphocyte, B and T cells; NRC (nucleated red cells), proerythroblast, basophilic erythroblast, polychromatophilic erythroblast, orthochromatic erythroblast; other, macrophages, stromal cells, reticular cells, osteoclastic cells, osteoblastic cells. No differences were seen between the overall percentages of various hematopoietic components in BM and rBM (p-value >0.05) confirming that the 3-D culture maintains the cellular composition of in vivo BM and can be used to evaluate the characteristics of all these types of cell as well as others that arise in bone marrow including heterogeneous malignant cell types. The only exceptions were a decreased proportion of monocytes in 3-D due to their probably differentiation to macrophages (p-value=0.015) and to osteoclasts, defined by an increased proportion of TRAP+ cells with osteoclastic morphology (p-value=0.001).

In FIG. 1c, the temporal organization of the 3-D culture was evaluated to better define the generation of rBM. BMC were grown in 3-D for the indicated number of days and the overall view of the culture analyzed using brightfield microscopy (200×). Black arrows indicate migrating cells, open arrows indicate hematopoietic cell colonies, and striped arrows indicate stromal cells. The 3-D culture was initiated from BMC suspended in ECM supplemented by MM serum; at day 0, BM cells are randomly distributed throughout the 3-D matrix. As early as day 1 in culture, cells begin to migrate within the ECM, marked by the appearance of leading edge protrusions. By day 4, colonies of hematopoietic cells localized to their respective niches, as discussed below. Stromal cells appeared at the endosteum after 7 days in culture and grew to cover the surface of the tissue culture vessel. By day 14, the architecture of the rBM closely resembled that of the in vivo BM. After 3-4 weeks, cells began to die.

FIG. 1d illustrates the proliferation of the various cellular compartments of the rBM after 3-D culture in accordance with various embodiments. Proliferation was measured by labeling BMC with carboxyfluorescein succinimidyl ester (CFSE) (n=4) and culturing in 3-D for the indicated number of days. Cells were isolated from 3-D, stained with fluorescent tagged antibodies and the proliferation capacity of the aggregate rBM (*p=0.001), CD19+B cells (*p=0.024, **p=0.006)), and CD138+PCs (*p=0.004) was measured by multicolor flow cytometry. In FIG. 1d, BM cells were labeled with CFSE (a cytoplasmic fluorescent dye that is reduced in intensity by 50% at each cell division) to determine whether the colonies in rBM reflected cell proliferation. The number of cell divisions was determined by measuring the intensity of fluorescence as a function of time. Non-dividing cells appear as bright green cells within the 3-D cultures as shown in FIG. 1d. Significant proliferation of the overall culture was observed between days 0 and 5, with cells continuing to proliferate through day 25 (not shown). Proliferation of the CD19+ B cells was very rapid from day 0 through day 5. Consistent with their lack of proliferation in vivo, CD138+ PCs divided more slowly than did B cells, and their proliferation only reached significance by day 25. Proliferation of cells within rBM combined with the proportionate preservation of hematopoietic compartments, as shown in FIG. 1b, implies that hematopoietic lineage relationships are preserved in the 3-D culture, teaching that theses cultures are of use for all kinds of malignant and nonmalignant hematopoietic conditions, including LRC and hematopoietic stem cells, and for malignant cells or cancer stem cells that spread from solid tumors into the bone marrow.

In an exemplary embodiment as illustrated in FIGS. 1a-d above, a method for preparing a culture apparatus for growth of eukaryotic cells in a multi-well tissue culture plate may comprise, for 20 wells of a 48 well plate (for other culture dishes adjust volumes accordingly):
1. Mixing 125 μl of fibronectin (1 mg/ml) with 36 μl of collagen I (3.5 mg/ml) for a 1:1 mixture of fibronectin to collagen I (5 μg of each protein/well).
2. Adding 1339 μl of PBS to the product of Step 1 and mix thoroughly (the “first mixture”).
3. Adding 65 μl/well of final mixture and spread evenly within the wells.
4. Incubating for at least 30 min at room temperature with occasional shaking to keep the surface coated at all times.
5. Mixing 1750 μl of Matrigel™ with 750 μl of fibronectin (1 mg/ml) for a 2:1 mixture of Matrigel™ to fibronectin (the “second mixture”). The mixture should be kept on ice in preparation for step 6.
6. Resuspending cells at 2.75×10⁵ cells/10 μl/well in PBS or Growth Medium.
7. (Optional) For proliferation analysis (cells should be protected from light following labelling),
   a. adding 0.25 μM CFSE prior to culture,
   b. mixing cells with dye and incubating at 40° C. for 15 min,
   c. adding 1 ml cold PBS and incubating on ice for 10 min,
   d. centrifuging and removing supernatant, and
   e. resuspending cells at plating density.
8. Removing coating mixture of steps 1-4 from wells, and adding 10 μl of the cell suspension from Step 6/7 to each coated well.
9. Allowing cells to settle/attach for 15 min in a 37° C., 5% CO₂ incubator.
10. Covering cells with 100 μl of Matrigel™/fibronectin mixture of Step 5.
11. Allowing matrix to solidify for 30 min in a 37° C., 5% CO₂ incubator.
12. Gently covering 3-D culture with 1 ml of media.

In another exemplary embodiment, a method for preparing a culture apparatus for growth of eukaryotic cells in a multi-well tissue culture plate comprises, for 20 wells of a 48 well plate (for other culture dishes adjust volumes accordingly):
1. Mixing 125 μl of fibronectin (1 mg/ml) with 36 μl of collagen I (3.5 mg/ml) for a 1:1 mixture of fibronectin to collagen I (5 μg of each protein/well).
2. Adding 1339 μl of PBS to the product of Step 1 and mix thoroughly (the “first mixture”).
3. Adding 65 μl/well of final mixture and spread evenly within the wells.
4. Incubate for at least 30 min at room temperature with occasional shaking to keep the surface coated at all times.
5. Mixing 1750 μl of Matrigel™ with 750 μl of fibronectin (1 mg/ml) for a 2:1 mixture of Matrigel™ to fibronectin. The mixture should be kept on ice in preparation for step 6.
6. Resuspending cells at 0.5×10⁶ cells/10 μl/well of PBS or Growth Medium
7. (Optional) For proliferation analysis (cells should be protected from light following labeling),
   a. adding 0.25 μM CFSE prior to culture,
   b. mixing cells with dye and incubating at 40° C. for 15 min,
   c. adding 1 ml cold PBS and incubating on ice for 10 min,
   d. centrifuging and removing supernatant, and
   e. resuspending cells at plating density.
8. Adding cells from Step 6/7 to the matrix at 0.5×10⁶ cells/100 μl of the mixture of Step 5 and mix very well (the “second mixture”).
9. Adding 100 μl of cell/matrix mix of Step 8 to each well.
10. Allowing matrix to solidify for 30 min in a 37° C., 5% CO₂ incubator.
11. Gently covering 3-D culture with 1 ml of media.

Persons with ordinary skill in the art will readily understand that these exemplary methods may be modified to produce desired results. In some embodiments the second mixture lacks cells when initially added to the wells, while in other embodiments the second mixture includes cells. Labeled cells, normal cells, cancer cells, bone marrow cells and other cell types may be added to the second mixture, alone or in combination. Additionally, growth media may be added to the first, second, and/or third mixture.

One skilled in the art will also realize that the optimal density of cells re-suspended and thereafter added to the individual wells is particular to each individual cell line. Therefore, the density listed in the steps above is to be varied through regular experimentation with a variety of densities. Embodiments contemplate a variety of densities of cells for inclusion in the 3-D culture apparatuses and methods. Embodiments also contemplate the optional addition of additives including but not limited to anti-viral compounds, antibacterial compounds, antifungal compounds, or media supplements, all of which are useful for encouraging the growth of cells of interest and discouraging the growth of cells, virus, or organisms not of interest.

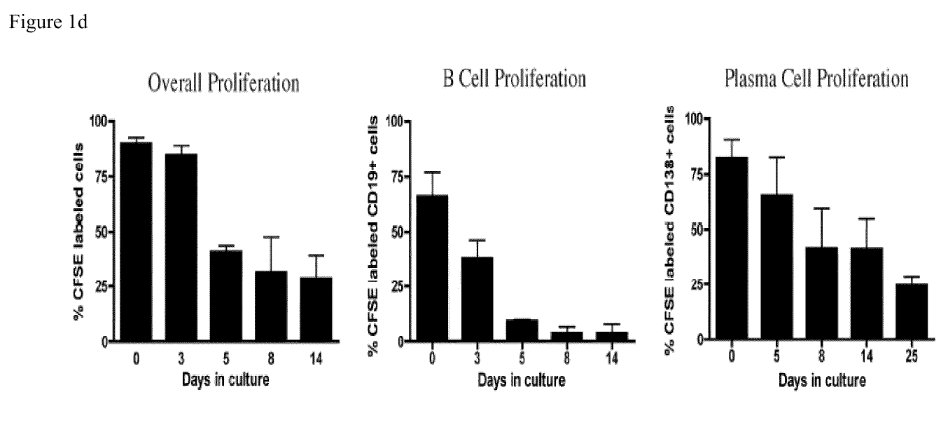

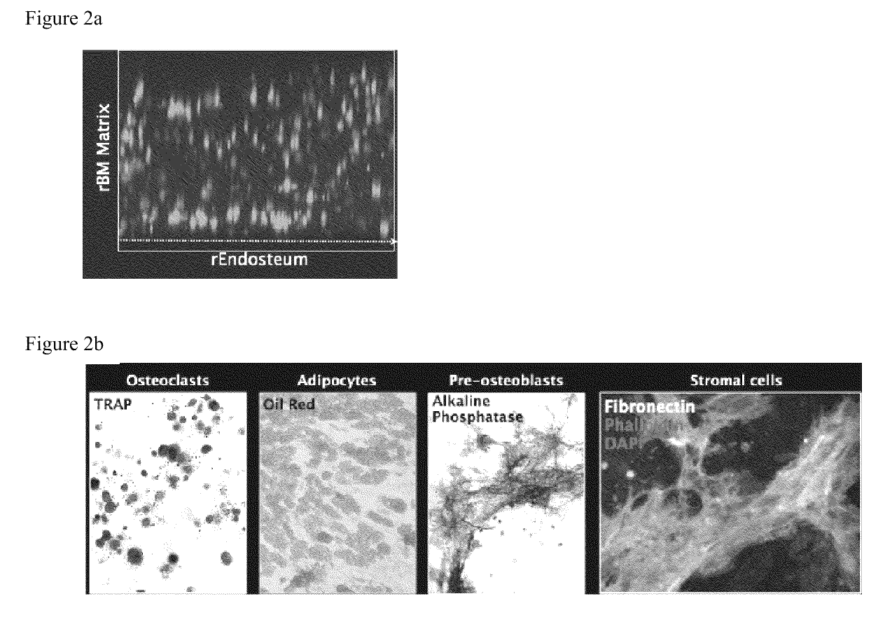

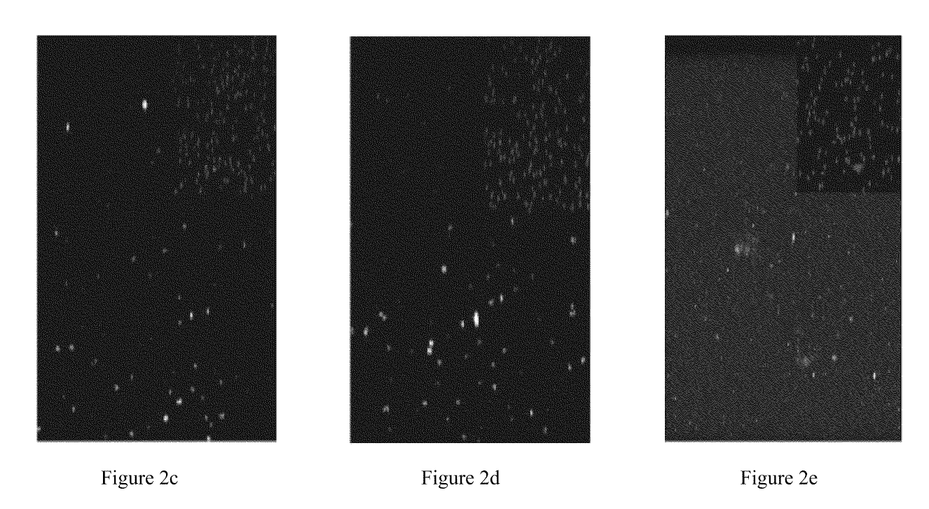

FIGS. 2a-d illustrate the maintenance of the architecture of in vivo BM by rBM in accordance with various embodiments. Physical stratification of the BM in vivo establishes distinct niches where dormant hematopoietic progenitor cells (HPC) are found at the bone surface in close association with the endosteum. More differentiated hematopoietic cells such as B cells and PCs may be found in the central marrow, with the most differentiated cells localized furthest away from the endosteum. The interaction between HPC, endosteal osteoblasts and osteoclasts, bone forming and resorbing cells respectively, provides for the maintenance of the HPC niche and mobilization of stem cells confirming the utility of the method for tests involving these and other hematopoietic cell types or cell types that home to the bone marrow.

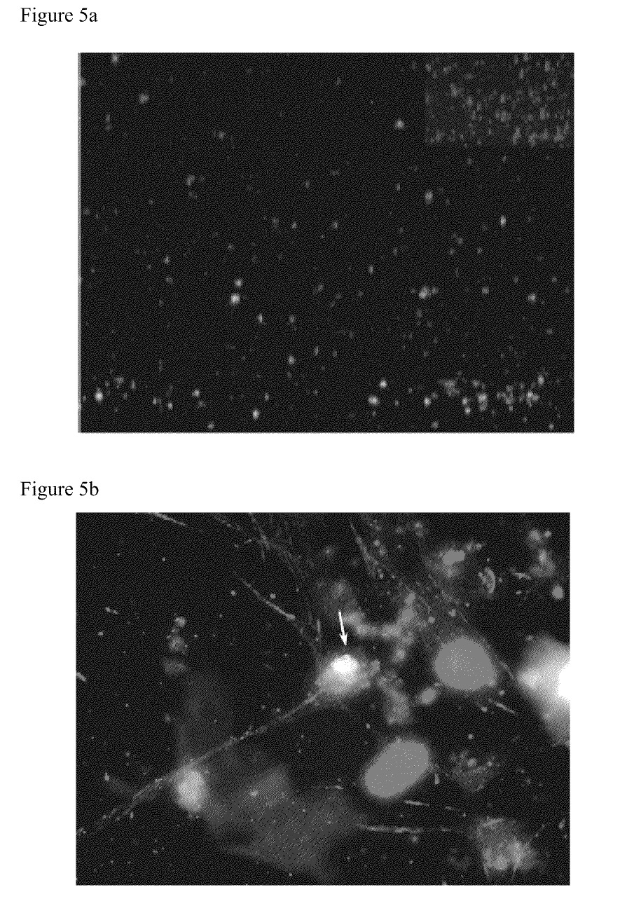

FIG. 2a illustrates the stratification of a 3-D culture in accordance with various embodiments. BMC were grown in rBM for 21 days and stained with DAPI (pseudocolored red) to mark nuclei, and the stratification of the 3-D culture was assessed by confocal microscopy as a 3D reconstruction from the confocal stack (100×). In 3-D culture, after completion of redistribution and proliferation, rBM may be stratified into approximately three layers, as shown in FIG. 2a. Similar to the architecture of the in vivo BM, the endosteal niche of rBM was composed of fibroblastic stromal cells, adipocytes, osteoclastic and osteoblastic cells residing at the rEnd (FIG. 2b in contact with CD34+ HPC (FIGS. 2c and 5b). CD19+ B cells were localized mainly to the middle layer and CD138+ PCs were mostly at the top layer of the rBM (FIG. 2c). Top to bottom, B cells comprised 6%, 10%, and 8% and PCs 24%, 11%, and 6% of the cells in each layer of the 3-D culture. Exclusion of the rEnd coating from the culture greatly reduced the stromal compartment (not shown).

FIG. 2b illustrates the redistribution of BM stromal cells to rEnd in a 3-D culture in accordance with various embodiments. 3-D cultures were grown as in (a), the rBM layer was removed and the cells at rEnd were stained with markers to identify stromal components observed at rEnd (adipocytes, Oil Red; fibroblasts, fibronectin and phallodin; osteoblasts, alkaline phosphatase (ALP) and Alizarin Red (supplemental FIG. 1); osteoclasts, tartarate-resistant acidic phosphatase (TRAP)). Brightfield microscopy (200×).

FIGS. 2c, d, and e show the positions of stem cells (CD34+CD45low), B cells (CD19+/CD33−), and PCs (CD138+/CD38+), respectively, in rBM after 3-D culture in accordance with various embodiments. Cells were sorted, labeled with CFSE (green) and co-cultured with unlabeled BMC. After 14 days in culture, rBM was stained with DAPI (blue inset) and the positions of the CFSE-labeled cells were analyzed by confocal microscopy (100×)

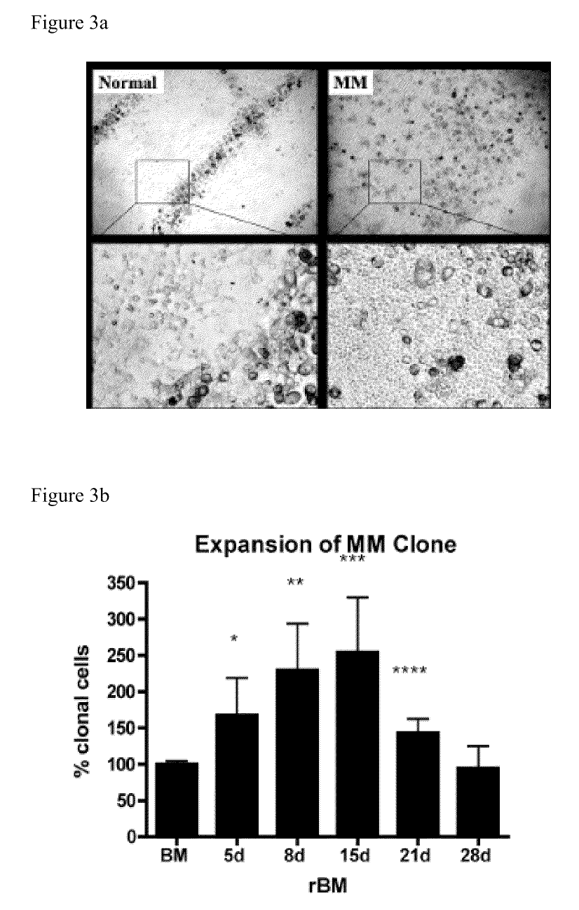

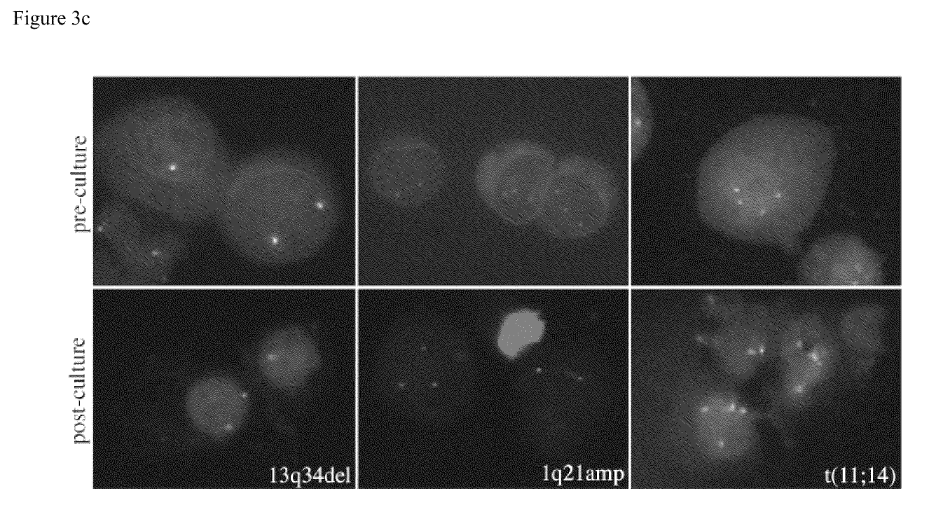

FIGS. 3a-c show the abnormal tissue architecture of MM rBM, exhibiting clonal expansion of MM cells with chromosomal abnormalities observed after 3-D culture, in accordance with various embodiments.

FIG. 3a shows BMC from normal donors and MM patients (n=3) grown in rBM for 21 days followed by assessment of the overall culture architecture by brightfield microscopy (top-50×, bottom-200×). Cells localized to “tracks” are TRAP+osteoclasts. BM cultures from normal donors were well-organized with osteoclasts and hematopoietic cells occupying distinct positions in the ECM.

In contrast, rBM from MM patients is disorganized in 3-D culture, with osteoclast-like cells intermingling with hematopoietic cells, as seen in FIG. 3a. 3-D cultures of sequential BM aspirates from a patient who progressed from plasmacytoma to MM highlight these architectural differences. rBM formed by the BMC from the plasmacytoma BM, taken from a site distant to the tumor, was organized with tracks of osteoclastic cells localized to areas distinct from the hematopoietic compartment. This organization was lost in the rBM formed by the BMC from the same patient after progression to MM.

FIG. 3b illustrates the extent of the malignant outgrowth of a MM clone in a 3-D culture as measured by RQ-PCR using patient specific primers. BMC (n=16) were grown in rBM for the indicated number of days. Each malignant cell has only one copy of the IgH VDJ rearranged template; RQ-PCR was used to determine the number of rearranged IgH VDJ templates as compared to a B2M standard curve and a IgH VDJ positive control curve. Percent clonal cells corresponds to the percent clonal VDJ templates present in the sample normalized to the μ2 microglobulin gene (*p=0.0065, **p=0.0001, ***p=0.0002, ****p=0.0034). This figure shows that rBM supports the expansion of the MM clone as measured by real-time quantitative PCR (RQ-PCR) analysis of cells harvested from 3-D, using patient-specific primers to amplify the clonotypic Ig heavy chain (IgH) VDJ gene rearrangement. The IgH VDJ rearrangement provides a unique clonal marker to identify all cells related to the malignancy. By day 15 there was a 2.54 fold increase in the percentage of the malignant cells in rBM compared to the pre-cultured BMC (p=0.0002). Sequencing of the PCR products confirmed that the IgH VDJ of MM cells arising in 3-D was identical to that of ex-vivo MM PC from the same patient.

FIG. 3c shows the result of fluorescence in situ hybridization (FISH) analysis of cells harvested from a 3-D culture in accordance with embodiments. MM appears to arise through progressive acquisition of complex chromosomal abnormalities. To confirm that PCs proliferating in 3-D cultures were a part of the MM clone, the chromosomal abnormalities in cultured PCs were compared to those confirmed to be present in ex-vivo PCs from the same patient, using fluorescence in situ hybridization (FISH). Cells from 3-D cultures were screened for the chromosomal abnormalities detected in pre-culture BM PC, using cytospins from the matching 21 day rBM (n=5) stained by interphase FISH. Left panel —PC with a deletion of 13q34 locus (1 green signal, arrow) and a normal cell (2 green signals). Middle panel —PC with an amplification of 1q21 (3 red signals, arrow) and a normal cell (2 red signals). Right panel—PC with t(11;14) translocation with 2 derivative chromosomes (2 yellow signals, short arrow), 1 normal chromosome 11 (1 red signal) and 1 normal chromosome 14 (1 green signal) (630×).

The compartment of PCs with chromosomal abnormalities matching pre-culture BM persisted in 3-D culture and had expanded 1.36 times by day 15 in culture. As measured by RQ-PCR, the aggregate number of MM cells at dl 5 of 3-D culture was 2.5 times greater than the number in pre-culture BM, as shown in FIG. 3b, suggesting that in addition to PC, other clonotypic MM compartments were also expanded in rBM.

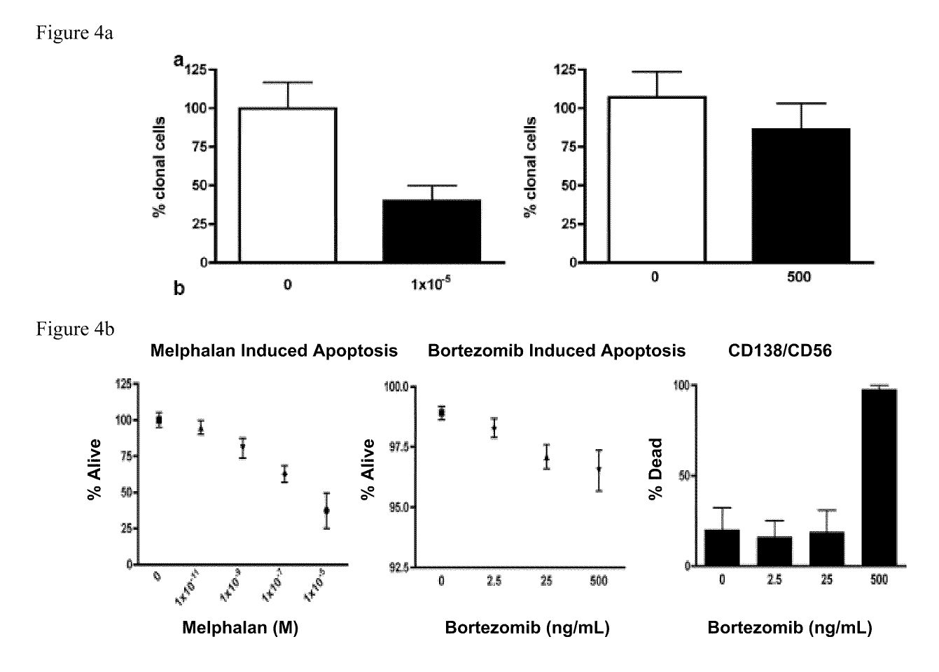

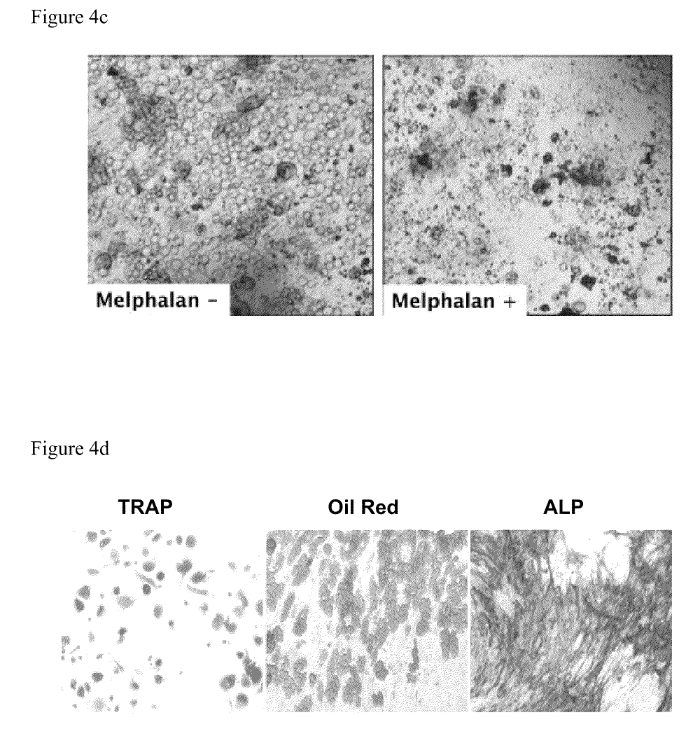

FIGS. 4a-d illustrate a comparative evaluation of two MM therapeutics in a 3-D BM cell culture assembly in accordance with various embodiments. Results show that melphalan and bortezomib affect the hematopoietic, but not the stromal compartment of rBM. MM BMC were grown in 3-D culture for 14 days as described for FIG. 1. An aliquot of melphalan or bortezomib was then added in fresh growth medium to each tested 3-D culture and incubated for seven days. Subsequently, fresh medium was added and 3-D cultures were incubated seven days to allow cells to recover from the treatments. Cells were then isolated using Cell Recovery Solution (obtained from BD Biosciences) by the manufacturer's method.

FIG. 4a illustrates the reduction of the clonal cells as measured by RQ-PCR with patient specific primers (melphalan p=0.016, bortezomib p=0.39). Pre-clinical testing of MM therapeutics has been hindered by the lack of systems to sustain growth of the MM clone. Since rBM contains the full complement of BM cells found in vivo, chemotherapeutic (melphalan) or biologically-based (bortezomib) agents were used to validate the 3-D model for pre-clinical use. Treatment of rBM with melphalan led to a 60% decrease in the number of clonotypic cells as measured by RQ-PCR.

FIG. 4b illustrates apoptosis within rBM as assessed by flow cytometry measuring AnnexinV reactivity post treatment with melphalan (left panel) or bortezomib (middle panel). MM specific cell killing by bortezomib was monitored in the CD138+/CD56+ population by AnnexinV reactivity (right panel).

FIG. 4c shows cell kill as monitored by brightfield microscopy (200×). rBM was grown and treated with melphalan as in FIG. 1a. Consistent with its myeloablative properties, melphalan induced apoptosis of all cell types within the hematopoietic hierarchy present in the rBM (as seen in FIGS. 4b and 4c), but the stromal compartment of the melphalan-treated rBM remained intact (see FIG. 4c). Bortezomib induced neither a significant reduction in clonotypic cells (see FIG. 4a) nor extensive apoptosis (see FIG. 4b) in rBM. Closer examination revealed that the only cell population subject to bortezomib induced apoptosis were CD138+CD56+ PC (FIG. 4b) accounting for a small increase in the overall apoptosis and a marginal reduction in clonotypic cells.

FIG. 4d shows cells at rEnd stained with TRAP (osteoclasts), Oil Red (adipocytes) and ALP (osteoblasts) after removal of the rBM layer. Brightfield microscopy (200×). Neither CD19+, CD138+, CD56+, CD33+, CD3+ cells of the hematopoietic compartment (data not shown) nor the stromal compartment were affected by bortezomib.

A reduction in BM plasmacytosis and the disappearance of monoclonal Ig coupled with relatively few side effects, coupled with inevitable relapse, indicate that bortezomib treatment targets a PC population responsible for disease symptoms while leaving other compartments intact. The agreement between clinical data and the pre-clinical studies in rBM validates the 3-D model as relevant to events in vivo. However, given the inevitable relapse of MM and the likelihood that MM-CSC escape current modes of therapy, a valid pre-clinical model should also enable monitoring the impact of therapy on cancer stem cells.

FIGS. 5a-b show the redistribution of cells including putative MM-CSC (label retaining cells, or LRC) to rEnd in a 3-D BM cell culture in accordance with various embodiments. Although residual disease almost certainly plays a role in relapse after high dose chemotherapy and stem cell rescue, the mobilized blood autograft has also been shown to harbor malignant cells capable of regenerating MM. Mobilized blood cells (MBC) from MM patients reconstitute a BM-like microenvironment in 3-D cultures and generate clonotypic MM cells. A defining property of cancer stem cells is their proliferative quiescence, which was measured here by the retention of CFSE fluorescent label (termed label retaining cells or LRC), which is lost when cells proliferate. To identify putative MM-CSC, focus was on cells from 3-D cultures of MBC that remained quiescent throughout the 3-D culture period. BMC were labeled with CFSE (green) and cultured for 21 days.

FIG. 5a shows rBM stained with DAPI (blue inset) to detect all cells in the cultures. The positions of the brightly staining CFSE+ LRCs were analyzed by confocal microscopy (100×).

FIG. 5b shows cells at rEnd after removal of the rBM layer. The cells at rEnd were fixed and stained with N-cadherin. CFSE+LRCs (green) are shown in contact with N-cadherin+ stromal cell (red), with DAPI stained nuclei (blue). LRC comprised 22% of the cells proximal to rEnd (FIG. 5a) in close contact with osteocalcin (not shown) and N-cadherin expressing osteoblastic cells (FIG. 5b). Overall, 60% of LRC localized to the endosteal niche, which has been shown to maintain dormancy of normal HPC.

FIGS. 6a-d illustrate the stem cell potential of LRCs in a 3-D BM cell culture assembly in accordance with various embodiments. Mononuclear cells were isolated from the mobilized blood preparations, labeled with CFSE, grown in 3-D for 14 days and treated with melphalan.

FIG. 6a illustrates the flow cytometry profile of the LRCs. The gate for sorting was set to isolate CFSE-high cells and the percentage of LRCs was calculated as the percent of CFSE-high cells in the live cell fraction of the 3-D culture.

FIG. 6b shows May-Grunwald Giemsa (MGG) staining of the sorted LRCs (top) and immunohistochemical staining of sorted LRC for CD20 (bottom).

FIG. 6c shows a representative colony from the colony forming unit (CFU) assay.

FIG. 6d illustrates the quantification of the LRCs as a percentage of total cells in the culture, CFUs as a percentage of LRCs, and clonal CFUs as a percentage of total colonies obtained in the CFU assay.

LRCs were drug-resistant as defined by their persistence in 3-D cultures treated with melphalan and showed 50% enrichment in the melphalan treated cultures consistent with the depletion in 3-D of other hematopoietic cells by melphalan by 50% or more, as show in FIG. 6a. LRCs were isolated from 3-D culture and sorted for bright expression of CFSE (FIG. 6a). The major LRC population (21%) was composed of lymphocytes as assessed by May-Grunwald Giemsa staining and confirmed as B lymphocytes based on their CD20 expression (see FIG. 6b). Non-lymphocyte LRC were terminally differentiated myelocytes, bands, polymorphonuclear cells, monocytes, or were other non-lymphocytic cells. No PC were detected among LRC. Sorted LRC were transferred to conventional carboxymethyl cellulose cultures normally used for counting colony forming units (CFU) to determine the number of CFU in the population. Sorted LRCs were placed into the CFU assay and the resulting colonies (shown in FIG. 6c) were individually harvested and assessed for the MM clonotypic signature, as measured by RQ-PCR with patient specific primers (FIG. 6d).

FIG. 7 illustrates the importance of serum or plasma from patients with MM or other cancers as a component of 3-D BM reconstruction cultures in accordance with various embodiments; HuN=normal human plasma, HuMM=myeloma plasma or serum, and FBS=fetal bovine serum.

CFU were found among LRC from untreated and treated culture, giving rise to colonies, about half of which gave rise to clonotypic progeny. Based on quantitation by patient-specific RQ-PCR, all progeny within a given colony were either 100% clonotypic or had no clonotypic cells, indicating that once transformed, LRC are committed to the myeloma lineage. The number of cells per colony was quantified by RQ-PCR for B2M. Self-renewal potential of LRCs was demonstrated in serial replating experiments where colonies formed by the LRCs were passaged to a secondary CFU assay to detect putative self-renewal capabilities. Although LRC clearly include normal HPC, they also include cells with characteristics predicted for MM-CSC, including quiescence, localization to rEnd, drug resistance and ability to generate clonotypic progeny. Clinically, this assay may assess the treatment efficacy by measuring the number of LRCs able to form clonotypic CFUs, and thus, predicted to underlie relapse. Taken together, these results demonstrate that 3-D culture of BM or MB maintains putative MM-CSC in a dormant state, from which they may be harvested and released from dormancy by CFU culture conditions. Therapeutic reductions in the number of CFU from 3-D LRC may provide a clinically feasible means to monitor the impact of new drugs on cancer stem cells from MM and other malignancies of the hematopoietic system.

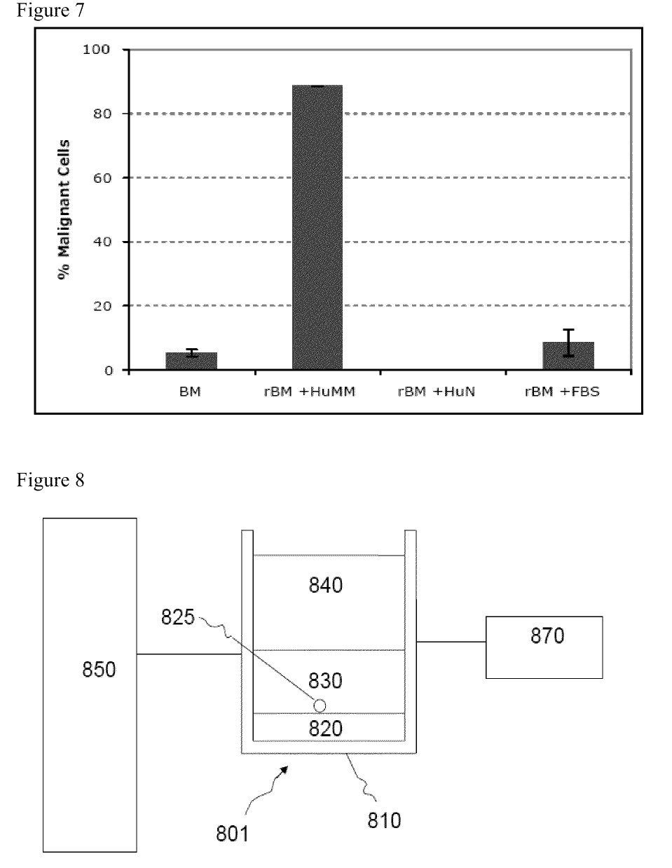

FIG. 8 illustrates an apparatus for 3-D culture in accordance with embodiments. In the illustrated embodiment, a 3-D tissue culture assembly 801 comprises a tissue culture vessel 810, a bottom layer 820 coupled to a surface of the tissue culture vessel 810 and comprising fibronectin and collagen, a middle layer 830 disposed on the bottom layer 820 and comprising a biological matrix mimetic and fibronectin, a top layer 840 disposed on the middle layer 830 and comprising a fluid derived from a vertebrate with cancer or nonmalignant disease of the bone marrow or blood, and a eukaryotic cell 825 disposed in or below the middle layer 830. The thickness, depth and/or dimensions of the bottom layer, middle layer and/or top layer may vary among embodiments. 3-D tissue culture assembly 801 may be coupled to an incubator 850, whether directly or indirectly coupled, to provide at least a source of heat for assembly 801. In the illustrated embodiment, 3-D tissue culture assembly 801 is also coupled to an imaging apparatus 870.

Some embodiments may comprise only a 3-D tissue culture assembly and an incubator, while other embodiments may further comprise an imaging apparatus. In some embodiments an imaging apparatus comprises a microscope. In embodiments, an imaging apparatus may include an automated imager, a digital microscope, and/or a flow cytometer. In some embodiments, an automated imager may be an automated confocal microscope. In embodiments, one or more cells may be conveyed from the 3-D tissue culture assembly to the imaging apparatus by an individual, by a device, and/or by an automated process. In embodiments of a 3-D tissue culture apparatus, an automated imager may be coupled to a digital microscope and/or a flow cytometer, and/or a digital microscope may be coupled to a flow cytometer. In embodiments, any or all of these components may be coupled to and/or controlled through a computer and/or a user interface. In embodiments adapted for high-throughput and/or high-content screening, control of some or all components may be automated.

Embodiments presented herein also provide methods for obtaining cancer stem cells and/or progenitor cells related to other diseases/conditions. In some embodiments, removal of cells from the 3-D tissue culture assembly may be accomplished by using a commercially available product such as BD™ Cell Recovery Solution or other similar product to depolymerize the second/middle gel layer. In other embodiments, removal of cells from the 3-D tissue culture assembly may be accomplished by treating a portion of the 3-D tissue culture assembly with an enzyme to depolymerize the second/middle layer. In embodiments, cells may be concentrated and/or isolated by centrifugation and/or washed with a cell culture medium, cell culture media and/or diluent. In some embodiments, the removed cells may be sorted using a flow cytometer to isolate cancer stem cells and/or other progenitor cells. In embodiments, sorted cells may be labeled with a fluorescent marker that indicates proliferation, such as CFSE. In some embodiments, cells may be removed from the 3-D tissue culture assembly without counting/sorting with a flow cytometer and/or without labeling.

Cells within a 3-D tissue culture assembly may be removed and added to secondary tissue culture assemblies, with each secondary tissue culture assembly comprising a tissue culture vessel and a cell culture substrate adapted for colony-forming cell growth, the cell culture substrate disposed within the tissue culture vessel. The secondary tissue culture assemblies may then be incubated and observed for colonies, which may be formed by the clonal expansion of a cancer stem cell or other progenitor cell. In embodiments, the cell culture substrate may be a solid, a semi-solid, a liquid or a gel, and may be adapted for the growth of a particular colony-forming cell type. In some embodiments, secondary tissue culture assemblies are formed using Petri dishes, while in other embodiments, secondary tissue culture assemblies are formed using microtiter plates. In embodiments, a secondary tissue culture assembly may be formed in any suitable tissue culture vessel. Additionally, in some embodiments, a therapy may be added to a 3-D tissue culture assembly and/or to a secondary tissue culture assembly in order to increase the proportion of therapy-resistant and/or drug-resistant cells in the resulting cell population.

Embodiments also provide methods for testing a therapy on a cell that originates from, homes to, and/or resides in the blood/bone marrow system. In an embodiment, a 3-D cell culture assembly as described above may be treated with a therapy, and subsequently cells from the 3-D tissue culture assembly may be removed from the assembly as described above. In some embodiments, the cells may be sorted and/or counted using a flow cytometer. Counting and/or sorting cells after exposure to the therapy may provide information as to the impact of the therapy on one or more cell types, on stem cells, on cancer cells, etc. In an embodiment, sorted and/or counted cells may be labeled either before or after their removal from the 3-D tissue culture assembly with a fluorescent marker that indicates proliferation, such as CFSE. In some embodiments, cells may be removed without counting/sorting with a flow cytometer and/or without labeling. In embodiments, cells previously exposed to a therapy in a 3-D tissue culture assembly may be subsequently added to a secondary tissue culture assembly and cultured as described above in order to determine the effects, if any, of the therapy on cells removed from the 3-D tissue culture and/or their progeny. For example, cells removed from a 3-D tissue culture assembly after treatment with a therapy may be counted with a flow cytometer to discover whether a cell type, such as a cancer stem cell, a normal cell, or a diseased cell, has been depleted by the treatment.

In embodiments, cells may be removed from a 3-D tissue culture assembly with or without previous addition of a therapy and/or with or without being counted, sorted and/or labeled. One or more of the removed cells may be added to a secondary tissue culture assembly, and a therapy may be added to the secondary tissue culture assembly in order to determine the effects, if any, of the therapy on the one or more cells removed from the 3-D tissue culture assembly and/or their progeny. For example, cells cultured in a secondary tissue culture assembly may be treated with a therapy, and the secondary tissue culture assembly may be examined to determine whether the treatment inhibits the formation of colonies.

Embodiments illustrated and discussed herein are suitable for the isolation and/or study of any cell type, including but not limited to blasts, polymorphonuclear cells, neutrophils, eosinophils, basophils, pre-PMN cells, promyelocytes, myelocytes, metamyelocytes, lymphocytes, B and/or T cells, nucleated red cells, proerythroblasts, basophilic erythroblasts, polychromatophilic erythroblasts, orthochromatic erythroblasts, macrophages, stromal cells, reticular cells, osteoclastic cells, osteoblastic cells, etc. Similarly, while some embodiments described with relation to methods, systems, and apparatuses for investigating the biological characteristics of, or effects of therapeutics on, MM/cancer cells, other embodiments provide corresponding systems, apparatuses and methods for any cell type and/or any type of cancer or disease found in the bone marrow or blood system, including but not limited to those listed above. Additionally, embodiments discussed herein are equally suitable for the investigation of both human and veterinary diseases/cells/therapies.

Although certain embodiments have been illustrated and described herein for purposes of description of the preferred embodiment, it will be appreciated by those of ordinary skill in the art that a wide variety of alternate and/or equivalent embodiments or implementations calculated to achieve the same purposes may be substituted for the embodiments shown and described without departing from the scope of the present invention. Those with skill in the art will readily appreciate that embodiments in accordance with the present invention may be implemented in a very wide variety of ways. This application is intended to cover any adaptations or variations of the embodiments discussed herein. Therefore, it is manifestly intended that embodiments in accordance with the present invention be limited only by the claims and the equivalents thereof.

What is claimed is:

1. A method for forming a 3-D eukaryotic cell culture, comprising:
coating at least a portion of a surface of a tissue culture vessel with a first mixture, the first mixture comprising fibronectin and collagen;
adding a second mixture onto at least the coated portion of the surface of the tissue culture vessel, the second mixture comprising fibronectin and a biological matrix mimetic;
adding a third mixture onto a surface of the second mixture, the third mixture comprising a fluid from a vertebrate subject having cancer or nonmalignant disease of the bone marrow or blood, wherein the tissue culture vessel contains at least one eukaryotic cell; and
incubating the tissue culture vessel.

2. The method of claim 1, wherein the eukaryotic cell is a bone marrow cell, a blood cell, or a stem cell.

3. The method of claim 1, wherein the eukaryotic cell is added to the second mixture before adding the second mixture onto at least the coated portion of the surface of the tissue culture vessel.

4. The method of claim 1, wherein the eukaryotic cell is added to the coated portion of the surface of the tissue culture vessel before adding the second mixture onto at least the coated portion of the surface of the tissue culture vessel.

5. The method of claim 1, wherein the fluid comprises one or more of plasma, serum, plasma from bone marrow, plasma from serum, peritoneal fluid, ascites fluid, cerebrospinal fluid, blood, lymph, and synovial fluid.

6. The method of claim 1, the second mixture further comprising a fluid from a vertebrate subject having cancer, the fluid comprising one or more of plasma, serum, plasma from bone marrow, serum from bone marrow, peritoneal fluid, ascites fluid, cerebrospinal fluid, blood, lymph, and synovial fluid.

7. The method of claim 1, wherein the eukaryotic cell is a label retaining cell.

8. The method of claim 1, wherein the eukaryotic cell is a putative multiple myeloma cancer stem cell.

9. The method of claim 1, wherein the tissue culture vessel is a microtiter plate.

10. The method of claim 1, wherein incubating the tissue culture vessel comprises incubating the tissue culture vessel at approximately 37° C. and approximately 5% CO₂.

11. The method of claim 3, wherein the second mixture forms a gel, and wherein the eukaryotic cell is embedded in the gel.

12. A method of obtaining a stem cell, comprising:
assembling a 3-D tissue culture assembly, the 3-D tissue culture assembly comprising
a first tissue culture vessel,
a bottom layer coupled to a surface of the first tissue culture vessel and comprising fibronectin and collagen,
a middle layer disposed on the bottom layer and comprising a biological matrix mimetic and fibronectin, the middle layer forming a gel,
a top layer disposed on the middle layer and comprising a fluid derived from a vertebrate, the vertebrate having cancer or nonmalignant disease of the bone marrow or blood, and
at least one eukaryotic cell, wherein the eukaryotic cell is disposed in or below the middle layer;
incubating the 3-D tissue culture assembly;
removing at least one eukaryotic cell from the 3-D tissue culture assembly;
adding the at least one removed eukaryotic cell to a secondary tissue culture assembly, the secondary tissue culture assembly comprising a second tissue culture vessel and a cell culture substrate adapted for culturing colony-forming cells, the cell culture substrate disposed within the tissue culture vessel; and
incubating the secondary tissue culture assemblies.

13. The method of claim 12, further comprising selecting a colony from the secondary tissue culture assembly that exhibits clonal expansion.

14. The method of claim 12, further comprising selecting a colony from the secondary tissue culture assembly that does not exhibit clonal expansion.

15. The method of claim 12, wherein said removing further comprises depolymerizing the middle layer to release at least one eukaryotic cell.

16. The method of claim 12, wherein said removing further comprises centrifugation of one or more layers of the 3-D tissue culture assembly.

17. The method of claim 12, further comprising adding a therapy to the 3-D tissue culture assembly to select for drug-resistant cancer stem cells.

18. The method of claim 12, further comprising adding a therapy to the secondary tissue culture assembly to select for therapy-resistant cancer stem cells.

19. The method of claim 12, wherein the eukaryotic cell is a stem cell.

20. The method of claim 12, wherein incubating the 3-D tissue culture assembly and incubating the secondary tissue culture assemblies comprises incubating at approximately 37° C. and approximately 5% CO₂.

21. An apparatus, comprising:
a 3-D tissue culture assembly, the assembly comprising
a tissue culture vessel,
a bottom layer coupled to a surface of the tissue culture vessel and comprising fibronectin and collagen,
a middle layer disposed on the bottom layer and comprising a biological matrix mimetic and fibronectin,
a top layer disposed on the middle layer and comprising a fluid derived from a vertebrate, the vertebrate having cancer or nonmalignant disease of the bone marrow or blood, and
at least one eukaryotic cell disposed in or below the middle layer; and
an incubator coupled to the assembly.

22. The apparatus of claim 19, further comprising an imaging apparatus coupled to the assembly.

23. The apparatus of claim 19, wherein the imaging apparatus includes an automated imager or a flow cytometer coupled to the assembly.
